(19) United States
(12) Patent Application Publication
(10) Pub. No.: US 2026/0035327 A1
(43) Pub. Date: Feb. 5, 2026

(54) ALKANEDIOL-CONTAINING COMPOSITION

(71) Applicant: DIC Corporation, Tokyo (JP)

(72) Inventors: Michihiro Tanaka, Sakura-shi (JP); Tatsuya Shigehiro, Sakura-shi (JP); Masanori Miyamoto, Sakura-shi (JP)

(73) Assignee: DIC Corporation, Tokyo (JP)

(21) Appl. No.: 19/269,701

(22) Filed: Jul. 15, 2025

(30) Foreign Application Priority Data
Jul. 30, 2024 (JP) 2024-123268

(51) Int. Cl. C07C 29/80

(52) U.S. Cl. CPC C07C 29/80

(57) ABSTRACT

Provided is an alkanediol-containing composition, subjected to the purification of alkanediols, that can reduce the clogging of pipes used in an alkanediol purification process and reduce energy required to reduce the clogging. An alkanediol-containing composition includes: an alkanediol having a melting point of 30° C. or more at normal pressure; and an alkanetriol having the number of carbon atoms of 3 to 8, in which the mass ratio of the alkanediol to the alkanetriol is 99.5:0.5 to 70:30.

### BACKGROUND OF THE INVENTION

### 1. Field of the Invention

The present invention relates to an alkanediol-containing composition.

### 2. Description of the Related Art

Alkanediols such as 1,6-hexanediol are useful intermediate products for the production of polymers such as polyesters and polyurethanes and polymer raw materials such as polycarbonate diols.

In an alkanediol purification process, water evaporation, distillation separation of low boiling point components, and distillation separation of high boiling point components are performed. Transportation between these distillation columns is performed through pipes. However, in the case of alkanediols having melting points of 30° C. or more at normal pressure, low temperatures in the pipes cause clogging of the pipes due to solidification of the alkanediols. In order to prevent the clogging of the pipes, it is necessary to keep the inside of the pipes at an adequate temperature, which requires energy.

In recent years, energy conservation has also been required in industrial processes. For example, Japanese Unexamined Patent Application Publication 2023-055396 has described an oil and fat extraction and distillation facility using flash steam to intend to save energy.

### SUMMARY OF THE INVENTION

As described above, the clogging of pipes due to solidification of alkanediols is a problem in the alkanediol purification process. In order to prevent the clogging of the pipes, the pipes are required to be heated, and energy is introduced for heating the pipes. On the other hand, lowering the melting point of the alkanediol-containing composition transported through the pipes in the purification facilities may be effective for preventing the clogging of the pipes. In addition, the energy required to heat the pipes may be reduced.

An object of the present invention, therefore, is to provide an alkanediol-containing composition that can be used for the purification of alkanediols, that can reduce the clogging of the pipes used in the alkanediol purification process and can reduce the energy required for reducing the clogging.

The present invention includes the following aspects.

(1) An alkanediol-containing composition including: an alkanediol having a melting point of 30° C. or more at normal pressure; and an alkanetriol having the number of carbon atoms of 3 to 8, in which the mass ratio of the alkanediol to the alkanetriol is 99.5:0.5 to 70:30.

(2) The alkanediol-containing composition according to (1), in which the alkanetriol includes an alkanetriol having a boiling point higher than the boiling point of the alkanediol at normal pressure.

(3) The alkanediol-containing composition according to (1) or (2), in which the alkanediol includes 1,6-hexanediol.

(4) The alkanediol-containing composition according to any one of (1) to (3), in which the alkanetriol includes at least one alkanetriol selected from the group consisting of 1,6,7-octanetriol, glycerin, 1,2,6-hexanetriol, and 1,2,8-octanetriol.

(5) The alkanediol-containing composition according to any one of (1) to (4), in which the alkanediol-containing composition includes a component produced by a living organism.

(6) The alkanediol-containing composition according to any one of (1) to (5), in which the alkanediol-containing composition includes a component derived from a biomass resource.

(7) The alkanediol-containing composition according to any one of (1) to (6) used for purification of alkanediols.

### BRIEF DESCRIPTION OF DRAWINGS

FIG. 1 is a schematic diagram illustrating an example of the equipment configuration of a purification process for purifying an alkanediol from an alkanediol-containing composition; and

FIG. 2 is a schematic diagram illustrating an example of the configuration of a distillation unit in the purification process for purifying the alkanediol from the alkanediol-containing composition.

### DETAILED DESCRIPTION OF EMBODIMENTS

### [Alkanediol-Containing Composition]

The first aspect of the present invention includes an alkanediol-containing composition including: an alkanediol having a melting point of 30° C. or more at normal pressure, and an alkanetriol having the number of carbon atoms of 3 to 8 (hereinafter may also referred to as the “alkanediol-containing composition”). In one embodiment, the mass ratio of the alkanediol to the alkanetriol (alkanediol: alkanetriol) in the composition is 99.5:0.5 to 70:30.

The alkanediol-containing composition can be used as a pre-purification composition of the alkanediol. A high-purity alkanediol can be obtained by purifying the alkanediol-containing composition. The alkanediol-containing composition may be a composition for alkanediol purification provided for purifying the alkanediol.

### <Alkanediol Having Melting Point of 30° C. or More at Normal Pressure>

The alkanediol-containing composition according to the present embodiment includes the alkanediol having a melting point of 30° C. or more at normal pressure (hereinafter referred to as an “alkanediol (A)”).

Alkanediols are compounds in which two hydrogen atoms of alkanes are replaced with two hydroxyl groups. The alkanediol (A) may be linear or branched, and a linear alkanediol (A) is preferable.

The “normal pressure” means atmospheric pressure (101 kPa).

Examples of the alkanediol (A) include alkanediols having the number of carbon atoms of 6 or more. The alkanediol (A) is preferably alkanediols having the number of carbon atoms of 6 to 15, more preferably alkanediols having the number of carbon atoms of 6 to 12, and further preferably linear alkanediols having the number of carbon atoms of 6 to 12.

Specific examples of the alkanediol (A) include 1,6-hexanediol (melting point 37 to 42° C.), 1,8-octanediol (melting point 57 to 61° C.), 1,9-nonanediol (melting point 46° C. to 49° C.), 1,10-decanediol (melting point 72 to 75° C.), 1,11-undecanediol (melting point 50 to 70° C.), and 1,12-dodecanediol (melting point 80 to 83° C.).

The alkanediol (A) may be singly or in combination of two or more.

The alkanediol (A) preferably includes at least one alkanediol selected from the group consisting of 1,6-hexanediol, 1,8-octanediol, 1,9-nonanediol, 1,10-decanediol, 1,11-undecanediol, and 1,12-dodecanediol and more preferably includes 1,6-hexanediol.

The alkanediol (A) may be a fossil fuel-derived component or a biologically derived component. The “fossil fuel-derived component” is a component produced from components included in fossil fuels such as petroleum. The “biologically derived component” is a living organism constituting component or a component produced using a biological reaction. Examples of the alkanediol (A) that is the biologically derived component include an alkanediol (A) produced by microbial reaction, an alkanediol (A) produced from components produced by microbial reaction as raw materials, and an alkanediol (A) produced from biomass resources as raw materials.

### <Alkanetriol Having Number of Carbon Atoms of 3 to 8>

The alkanediol-containing composition according to the present embodiment includes an alkanetriol having the number of carbon atoms of 3 to 8 (hereinafter referred to as an “alkanetriol (B)”). The melting point of the alkanediol-containing composition is lowered by including the alkanetriol (B) in the alkanediol-containing composition. This allows the occurrence of the pipe clogging in the purification process to be reduced.

Alkanetriols are compounds in which three of the hydrogen atoms included in alkanes are replaced with three hydroxyl groups. The alkanetriol (B) may be linear or branched, and a linear alkanetriol is preferable. The alkanetriol (B) is preferably a linear alkanetriol having the number of carbon atoms of 3 to 8.

Specific examples of the linear alkanetriol having the number of carbon atoms of 3 to 8 include, but are not limited to, glycerin (the number of carbon atoms of 3), 1,2,5-pentanetriol (the number of carbon atoms of 5), 1,3,4-pentanetriol (the number of carbon atoms of 5), 1,2,6-hexanetriol (the number of carbon atoms of 6), 1,4,5-hexanetriol (the number of carbon atoms of 6), 1,6,7-octanetriol (the number of carbon atoms of 8), 1,2,8-octanetriol (the number of carbon atoms of 8), and 1,3,8-octanetriol (the number of carbon atoms of 8).

Specific examples of the branched alkanetriols having the number of carbon atoms of 3 to 8 include, but are not limited to, 2-methyl-1,2,3-propanetriol (the number of carbon atoms of 4), 2-ethyl-2-hydroxymethyl-1,3-propanediol (the number of carbon atoms of 5), and 3-methylpentane-1,3,5-triol (the number of carbon atoms of 6).

The alkanetriol (B) may be singly or in combination of two or more.

The alkanetriol (B) is preferably a linear alkanetriol, preferably includes at least one alkanetriol selected from the group consisting of 1,6,7-octanetriol, glycerin, 1,2,5-pentanetriol, 1,2,6-hexanetriol, and 1,2,8-octanetriol, more preferably includes at least one alkanetriol selected from the group consisting of 1,6,7-octanetriol, glycerin, 1,2,6-hexanetriol, and 1,2,8-octanetriol, further preferably includes at least one alkanetriol selected from the group consisting of 1,6,7-octanetriol and glycerin, and particularly preferably includes 1,6,7-octanetriol.

The alkanetriol (B) preferably includes an alkanetriol having a boiling point higher than the boiling point of the alkanediol (A) at normal pressure. In other words, the boiling point of the alkanetriol (B) is higher than that of the alkanediol (A). In the alkanediol purification process, the distillation separation of the high boiling point components that have higher boiling points than that of the alkanediol is usually performed in the final step. When the boiling point of the alkanetriol (B) is higher than that of the alkanediol (A), the alkanetriol (B) remains in the alkanediol-containing composition until just before the final step of purification. Therefore, the pipe clogging can be reduced until the final process. The alkanetriol (B) is separated from the alkanediol (A) and removed by the distillation separation in the final step.

The alkanetriol (B) may be a fossil fuel-derived component or a biologically derived component. Examples of the alkanetriol (B) that is the biologically derived component include an alkanetriol (B) produced by microbial reaction, an alkanetriol (B) produced from components produced by microbial reaction as raw materials, and an alkanetriol (B) produced from biomass resources as raw materials.

In one embodiment, the mass ratio of the alkanediol (A) to the alkanetriol (B) (alkanediol (A):alkanetriol (B)) in the alkanediol-containing composition is 99.5:0.5 to 70:30. The mass ratio of the alkanediol (A) to the alkanetriol (B) within the above range allows a melting point lowering effect to be obtained to the extent that the pipe clogging can be reduced. In addition, in the case where a high-purity alkanediol (for example, a purity of 95% or more) is obtained by purification of the alkanediol-containing composition, a decrease in alkanediol yield can be reduced.

The mass ratio of the alkanediol (A) to the alkanetriol (B) in the alkanediol-containing composition is preferably 99:1 to 80:20, more preferably 98:2 to 90:10, and further preferably 97:3 to 93:7.

The content of the alkanediol (A) and the alkanetriol (B) in the alkanediol-containing composition can be measured by known methods. Examples of measurement methods include high-performance liquid chromatography (HPLC), gas chromatography (GC), and gas chromatography-mass spectrometry (GC-MS).

### <Other Components>

The alkanediol-containing composition may include other components in addition to the alkanediol (A) and the alkanetriol (B). Examples of other components include water, components produced by living organisms, and components derived from biomass resources.

(Components Produced by Living Organisms/Components Derived from Biomass Resources)

The alkanediol-containing composition may include components produced by living organisms and/or components derived from biomass resources.

The “biomass resources” are organic resources derived from biological constituent substances. The biomass resources may be resources derived from any of plants, animals, and microorganisms. Examples of preferable biomass resources include plant resources such as rice husk, rice bran, old rice, corn, sugarcane, cassava, soybean, okara (soy pulp), bagasse, vegetable oil, fats, used paper, paper residues, and bionaphtha. The biomass resources may be microbial cells. The “components derived from biomass resources” are components produced from the biomass resources as raw materials.

The alkanediol-containing composition may be a culture solution or culture supernatant of microorganisms that produce the alkanediol (A) (hereinafter may also referred to as “alkanediol-producing bacteria”). The alkanediol-producing bacteria may be, for example, genetically modified bacteria to which genes of enzymes catalyzing reactions in alkanediol (A) synthesis pathway are introduced. For example, a method has been reported in which alkanediol is produced by microorganisms such as coryneform bacteria and E. coli using biomass resources as substrates (Japanese Unexamined Patent Application Publication No. 2020-114227 and Japanese Unexamined Patent Application Publication (Translation of PCT Application) No. 2016-533162).

In the case where the alkanediol-containing composition is the culture solution or the culture supernatant of the alkanediol-producing bacteria, the culture solution may include the alkanetriol (B) as a by-product in addition to the alkanediol (A). The ratio of the alkanediol (A) to the alkanetriol (B) in the culture supernatant can be adjusted by adjusting the culture time and other factors. In the case where the mass ratio of the alkanediol (A) to the alkanetriol (B) in the culture supernatant is not in the range of 99.5:0.5 to 70:30, the mass ratio may be adjusted by adding the alkanediol (A) or the alkanetriol (B) to the culture supernatant.

In the case where the alkanediol-producing bacteria are genetically modified bacteria into which the genes of enzymes catalyzing the reactions of the synthetic pathway of the alkanediol (A) (hereinafter referred to as an “alkanediol synthase”) are introduced into E. coli or other bacteria, the genetically modified bacteria can be cultured in a medium including organic carbon sources, nitrogen sources, inorganic salts, and other substances. Examples of the organic carbon sources include, but are not limited to, sugars (for example, glucose, sucrose, and starch) and sugar alcohols (for example, glycerin, sorbitol, and maltitol). Examples of the nitrogen sources include, but are not limited to, enzyme extracts, amino acids, peptones, peptides, ammonium salts, nitrate salts, and nitrite salts. Examples of the inorganic salts include phosphates, potassium salts, and sulfates. In addition to the above components, the medium may include vitamins (for example, vitamin B12) and antibiotics (for example, carbenicillin, kanamycin, and chloramphenicol). The genetically modified bacteria can be cultured and proliferated under aerobic conditions. The culture temperature may be set to, for example, 20 to 40° C. As the culture time under the aerobic conditions becomes longer, the organic carbon sources in the medium are more consumed. For example, in the case where an alkanetriol such as glycerin is used as the organic carbon source, the amount of the remaining alkanetriol can be adjusted by controlling the culture time under the aerobic conditions.

The genetically modified bacteria may be induced to express alkanediol synthase. In the case where the alkanediol synthase gene is functionally linked to an inducible promoter, the expression of the alkanediol synthase gene can be induced by satisfying conditions under which the inducible promoter induces the gene expression. For example, in the case where a lacZ promoter is used as the promoter, the expression of the alkanediol synthase gene can be induced by adding isopropyl-β-thiogalactopyranoside to the medium. After the induction of the alkanediol synthase expression, the genetically modified bacteria may be cultured under the aerobic conditions in order to sufficiently express the alkanediol synthase. The culture time may be set to, for example, 1 to 5 hours. The culture temperature may be set to, for example, 20 to 40° C.

After expressing the alkanediol synthase, an additional carbon source may be added to the medium and the genetically modified bacteria are cultured under anaerobic conditions. The anaerobic conditions may be achieved, for example, by filling the incubator with nitrogen to form a nitrogen atmosphere. The culture time under the anaerobic conditions may be set to, for example, 5 hours or more and preferably 8 hours or more. As the culture time under the anaerobic conditions becomes longer, the ratio of the alkanetriol (B) to the alkanediol (A) becomes higher. Examples of the upper limit value of the culture time under the anaerobic conditions include 100 hours or less. The upper limit value is preferably 90 hours or less, more preferably 80 hours or less, further preferably 70 hours or less, and further more preferably 60 hours or less.

In the case where the alkanediol-containing composition is a culture solution of the alkanediol-producing bacteria, the culture solution may include components such as microbial cells, culture medium components, and microbial metabolites, in addition to the alkanediol (A) and the alkanetriol (B). These components may be the components produced by the living organisms and/or the components derived from the biomass resources.

### <Method for Purifying Alkanediol (A)>

The alkanediol-containing composition can be used as a composition for alkanediol purification in order to obtain the high-purity alkanediol (A). The high-purity alkanediol (A) (for example, 99.5% or higher) can be obtained by purifying the alkanediol-containing composition using a combination of one or more known purification methods. The alkanediol-containing composition may be an intermediate product obtained in the alkanediol purification process described below.

The alkanediol-containing composition can be purified to obtain the alkanediol (A) by, for example, one or more processes selected from the group consisting of a centrifugal separation process, a membrane separation process, a cation removal process, an anion removal process, a water removal process, a low boiling point component removal process, and a high boiling point component removal process.

### (Centrifugal Separation Process)

Examples of the centrifugal separation include centrifugal sedimentation and centrifugal filtration. In the centrifugal separation, the operating conditions are not particularly limited, and a centrifugal force of 100 G to 10,000 G is frequently used. In the membrane separation described below, particles such as microorganisms frequently clog the membrane. Therefore, the membrane separation process may be performed after particles such as microorganisms are coarsely removed by the centrifugal separation process.

### (Membrane Separation Process)

In the case where the alkanediol-containing composition includes solid components, the membrane separation process may be performed. In the case where the alkanediol-containing composition is the culture solution of the alkanediol-producing bacteria, the membrane separation process may be performed on the centrifugal supernatant after removing the bacterial cells by centrifugal separation or other means. Examples of the membrane separation process also include microfiltration, ultrafiltration, nanofiltration, or a combination thereof. The material of the membrane is not particularly limited. Examples of the material include organic films made of polyolefin, polysulfone, polyacrylonitrile, polyamide, and polyvinylidene fluoride and films of inorganic materials such as ceramics. The operation method can be performed using both dead-end type operation and cross-flow type operation.

The microfiltration can be performed, for example, in order to separate particles having a particle size in the range of about 0.05 to about 10 μm. Examples of equipment configurations for the microfiltration include cross-flow filtration using wound spirals, hollow fibers, or flat sheet (cartridge) microfiltration elements. The microfiltration may include filtration passed through a membrane having a pore diameter of about 0.05 to about 10 μm. The microfiltration membrane can have a molecular weight cutoff (MWCO) of about 20,000 Daltons or more. The term “molecular weight cutoff” is used in order to indicate the particle size that retains about 90% of the particles with the membrane.

The ultrafiltration is a selective separation method that uses a pressure of up to about 145 psi (10 bar) to pass through a membrane. Examples of the equipment configurations for the ultrafiltration include cross-flow filtration using wound spirals, hollow fibers, or flat sheet (cartridge) ultrafiltration elements. These elements may be composed of a polymeric membrane or a ceramic membrane having a molecular weight cutoff of less than about 200,000 Daltons.

The nanofiltration can be performed in order to remove high molecular weight impurities that cannot be removed by the microfiltration and/or the ultrafiltration or other processes. The nanofiltration may include filtration passed through a membrane having a molecular weight cutoff (MWCO) (a pore diameter of about 0.0005 to about 0.005 μm) of about 100 Daltons to about 2,000 Daltons. For example, the nanofiltration membrane can have a MWCO of about 100 to about 500 Daltons, about 100 to about 300 Daltons, or about 150 to about 250 Daltons. The nanofiltration is commonly operated at pressures from 70 psi to 700 psi, 200 psi to 650 psi, 200 psi to 600 psi, 200 psi to 450 psi, 70 psi to 400 psi, about 400 psi, about 450 psi, or about 500 psi.

The centrifugal separation process and the membrane separation process can be performed, for example, by appropriately combining the above known methods. For example, the ultrafiltration and the nanofiltration may be performed in this order. Alternatively, the centrifugal separation, the ultrafiltration, and the nanofiltration may be performed in this order. Alternatively, the microfiltration and the nanofiltration may be performed in this order.

### (Cation Removal Process)

The cation removal process can be performed by bringing the alkanediol-containing composition into contact with a cation exchange resin or by bringing the alkanediol-containing composition into contact with a cation exchange membrane and electrodialyzing with applied voltage. In the case of contact with the cation exchange resin, the contact may be performed, for example, by feeding the cation exchange resin into the alkanediol-containing composition placed in a suitable container and stirring the mixture. After the contact with the cation exchange resin, the cation exchange resin can be separated from the alkanediol-containing composition by filtration using a filter or other means. Alternatively, the alkanediol-containing composition may be brought into contact with the cation exchange resin by introducing the alkanediol-containing composition into a column packed with the cation exchange resin. The contact of the alkanediol-containing composition with the cation exchange resin can be performed, for example, under temperature conditions of 30 to 50° C. In the case where the electrodialysis is performed, the alkanediol-containing composition and brine are alternately fed between each membrane partitioned by cation-exchange membranes and anion-exchange membranes to perform the electrodialysis. Examples of the conditions for the electrodialysis include an initial current density of 0.5 to 15 A/dm² and a voltage of 0.1 to 1.5 V/tank.

### (Anion Removal Process)

The anion removal process can be performed by bringing the alkanediol-containing composition into contact with an anion exchange resin or by bringing the alkanediol-containing composition into contact with an anion exchange membrane and electrodialyzing with applied voltage. In the case of contact with the anion exchange resin, the anion removal process may be performed, for example, by feeding the anion exchange resin into an alkanediol-containing composition contained in a suitable container and stirring the mixture. After the contact with the anion exchange resin, the anion exchange resin can be separated from the alkanediol-containing composition by filtration using a filter or other means. Alternatively, the alkanediol-containing composition may be brought into contact with the anion exchange resin by introducing the alkanediol-containing composition into a column packed with the anion exchange resin. The contact between the alkanediol-containing composition and the anion exchange resin can be performed, for example, under temperature conditions of 30 to 50° C.

### (Water Removal Process)

The water removal process can be performed by distillation of the alkanediol-containing composition. The distillation can be performed using a distiller or distillation column. The distillation for water removal can be performed, for example, by setting the top temperature to 70° C. Water can be removed from the alkanediol-containing composition by discharging the distilled water out of the top of the distillation column and taking out the alkanediol-containing composition from the bottom.

### (Low Boiling Point Component Removal Process)

The low boiling point components are components having lower boiling points than that of the alkanediol (A). The low boiling point component removal process can be performed by distillation of the alkanediol-containing composition. The distillation can be performed using a distiller or distillation column. In the distillation for removing the low boiling point components, the column top temperature can be set depending on the boiling point of the alkanediol (A). For example, the top temperature can be set to a temperature of about 10 to about 20° C. lower than the boiling point of the alkanediol (A). The low boiling point components can be removed from the alkanediol-containing composition by discharging the distilled low boiling point components from the top of the distillation column and taking out the alkanediol-containing composition from the bottom.

### (High Boiling Point Component Removal Process)

The high boiling point components are components having higher boiling points than that of the alkanediol (A). The high boiling point component removal process can be performed by distillation of the alkanediol-containing composition. The distillation can be performed using a distiller or distillation column. In distillation for removing high boiling point components, the column bottom temperature can be set depending on the boiling point of the alkanediol (A). For example, the bottom temperature can be set to a temperature of about 10 to about 20° C. higher than the boiling point of the alkanediol (A). The high boiling point components can be removed from the alkanediol-containing composition by discharging the distilled alkanediol-containing composition from the top of the distillation column and taking out the high boiling point components from the bottom.

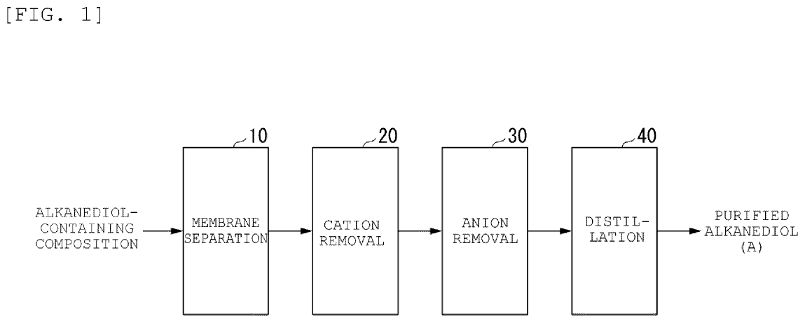

FIG. 1 is a schematic diagram illustrating an example of an equipment configuration in the purification process for purifying the alkanediol (A) from the alkanediol-containing composition. The purification process illustrated in FIG. 1 can be applied, for example, in the case where the alkanediol-containing composition is the culture solution or the culture supernatant of the alkanediol-producing bacteria.

In FIG. 1, the alkanediol-containing composition is processed by a membrane separation unit 10, a cation removal unit 20, an anion removal unit 30, and a distillation unit 40 to give the purified alkanediol (A). The membrane separation unit 10 performs the membrane separation process. The cation removal unit 20 performs the cation removal process. The anion removal unit 30 performs the anion removal process. The distillation unit 40 performs any one or more of the following processes: the water removal process, the low boiling point component removal process, and the high boiling point component removal process.

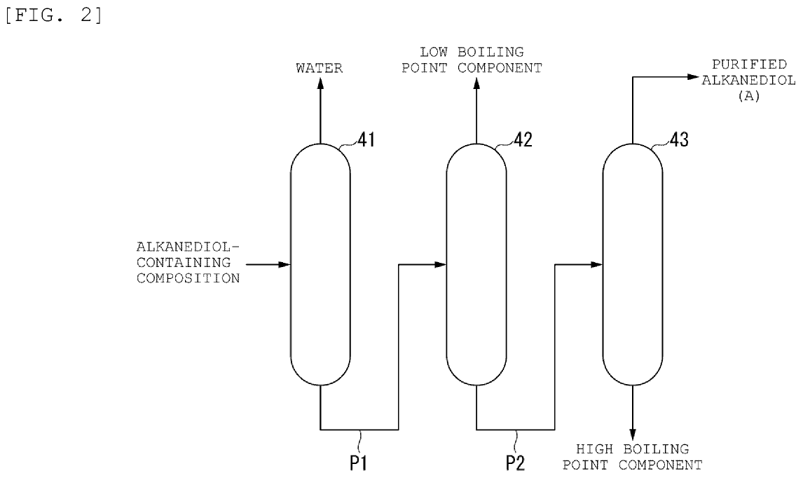

FIG. 2 is a schematic diagram illustrating an example of the configuration of the distillation unit 40. In FIG. 2, the alkanediol-containing composition is processed by a distillation column for water removal 41, a distillation column for low boiling point component removal 42, and a distillation column for high boiling point component removal 43 to give the purified alkanediol (A). The distillation column for water removal 41 performs the water removal process. The distillation column for low boiling point component removal 42 performs the low boiling point component removal process. The distillation column for high boiling point component removal 43 performs the high boiling point component removal process.

In the purification process illustrated in FIGS. 1 and 2, water can be included in the alkanediol-containing composition until before the water removal process by the distillation column for water removal 41. Including water in the alkanediol-containing composition allows the melting point of the alkanediol-containing composition to be lowered. Therefore, the risk of the pipe clogging is low in the pipes before the alkanediol-containing composition is fed to the distillation column for water removal 41. However, conventionally, there has been a high risk of the pipe clogging in pipes P1 and P2 after passing through the distillation column for water removal 41. The melting point of the alkanediol-containing composition according to the present embodiment is lowered by including a predetermined ratio of the alkanetriol (B) to the alkanediol (A). This allows the pipe clogging in the pipes P1 and P2 to be reduced.

As described above, the specific embodiments of the present invention have been described in detail. The present invention, however, is not limited to the embodiments described above. Various modifications, amendments, and combinations may be adopted for each configuration, element, and feature to the extent that they do not depart from the gist of the invention.

The respective terms “include” and “have” do not exclude the presence of elements other than the elements to which these words refer as objects, unless otherwise noted, and these terms are to be used interchangeably.

A numerical value range represented using “to” means a range that includes the numerical values described before and after “to” as the lower limit value and the upper limit value.

The contents of each literature referred to herein are hereby incorporated herein and made a part of the present specification.

### EXAMPLES

Hereinafter, the present invention will be described with reference to Examples. The present invention, however, is not limited to the following Examples.

### Examples 1 to 18, Comparative Examples 1 to 5

### <Preparation of Alkanediol-Containing Compositions>

The alkanediol component and each of the alkanetriol components listed in Tables 1 to 3 were mixed to prepare alkanediol-containing compositions. The values listed in Tables 1 to 3 represent mass ratios.

### <Evaluation of Degree of Melting Point Depression>

The melting points of the alkanediol and the alkanediol-containing composition were measured using a differential scanning calorimeter (DSC) (Hitachi High-Tech Science X-DSC7000) with the following temperature program.

Temperature program: First temperature rise −80° C. (20 min)→10° C./min→60° C. (1 min), First temperature fall 60° C. →10° C./min→−80° C. (20 min), Second temperature rise −80° C.→10° C./min→60° C. (1 min).

The degree of melting point depression (ΔT) was calculated from the measured melting point of the alkanediol (Tm_{D}) and the melting point of the alkanediol-containing composition (Tm_{D/T}) using the following formula.

$$Δ T = {Tm}_{D} - T {m}_{D / T}$$

The degree of melting point depression was evaluated according to the following evaluation criteria and listed in Tables 1 to 3 as “Degree of melting point depression”.

### Evaluation Criteria:

A: ΔT is 10.0° C. or more.
B: ΔT is 1.0° C. or more and less than 10.0° C.
C: ΔT is 0.2° C. or more and less than 1.0° C.
D: ΔT is less than 0.2° C.

### <Evaluation of Alkanediol Yield in Final Purified Product>

Components having a higher boiling point than that of the alkanediol in the alkanediol-containing composition were removed with a continuous distillation column. An Oldershaw distillation column was used as the continuous distillation column. The alkanediol-containing composition was fed to the distillation column and a column bottom temperature was controlled to be constant at 260° C. High boiling point components in the alkanediol-containing composition were removed by continuous removal from the bottom of the column. The column distillate was obtained from the top of the column. The column distillate was analyzed by gas chromatography (GC (GC7890B) manufactured by Agilent Technologies Japan, Ltd.) and a final purified product having an alkanediol purity of 99.5% or more was obtained. The yield of the alkanediol in the final purified product was calculated from the amount of the alkanediol in the alkanediol-containing composition before purification and the amount of the alkanediol in the final purified product.

The yield of the alkanediol was evaluated according to the following evaluation criteria and listed in Tables 1 to 3 as “Yield”.

### Evaluation Criteria:

A: Yield is 80% or more.
B: Yield is 75% or more and less than 80%.
C: Yield is less than 75%.

TABLE 1

| | | | | | | | | Comparative | |
|---|---|---|---|---|---|---|---|---|---|
| | | Example | | | | | | Example | |
| Component | | 1 | 2 | 3 | 4 | 5 | 6 | 1 | 2 |
| Alkanediol | 1,6-Hexanediol | 99.5 | 99.0 | 95.0 | 90.0 | 80.0 | 70.0 | 99.9 | 60.0 |
| Alkanetriol | 1,6,7-Octanetriol | 0.5 | 1.0 | 5.0 | 10.0 | 20.0 | 30.0 | 0.1 | 40.0 |
| | Glycerin | — | — | — | — | — | — | — | — |
| | 1,2,6-Hexanetriol | — | — | — | — | — | — | — | — |
| | 1,2,8-Octanetriol | — | — | — | — | — | — | — | — |
| Evaluation | Degree of | B | B | A | A | A | A | C | A |
| | melting point | | | | | | | | |
| | depression | | | | | | | | |
| | Yield | A | A | A | B | B | B | A | C |

[Mass ratio]

TABLE 2

| | | | | | | | | Comparative | |
|---|---|---|---|---|---|---|---|---|---|
| | | Example | | | | | | Example | |
| Component | | 7 | 8 | 9 | 10 | 11 | 12 | 3 | 4 |
| Alkanediol | 1,6-Hexanediol | 99.5 | 99.0 | 95.0 | 90.0 | 80.0 | 70.0 | 99.9 | 60.0 |
| Alkanetriol | 1,6,7-Octanetriol | — | — | — | — | — | — | — | — |
| | Glycerin | 0.5 | 1.0 | 5.0 | 10.0 | 20.0 | 30.0 | 0.1 | 40.0 |
| | 1,2,6-Hexanetriol | — | — | — | — | — | — | — | — |
| | 1,2,8-Octanetriol | — | — | — | — | — | — | — | — |
| Evaluation | Degree of | B | B | B | A | A | A | C | A |
| | melting point | | | | | | | | |
| | depression | | | | | | | | |
| | Yield | A | A | A | B | B | B | A | C |

[Mass ratio]

TABLE 3

| | | | | | | | | Comparative |
|---|---|---|---|---|---|---|---|---|
| | | Example | | | | | | Example |
| Component | | 13 | 14 | 15 | 16 | 17 | 18 | 5 |
| Alkanediol | 1,6-Hexanediol | 99.5 | 95.0 | 70.0 | 99.5 | 95.0 | 70.0 | 100 |
| Alkanetriol | 1,6,7-Octanetriol | — | — | — | — | — | — | — |
| | Glycerin | — | — | — | — | — | — | — |
| | 1,2,6-Hexanetriol | 0.5 | 5.0 | 30.0 | — | — | — | — |
| | 1,2,8-Octanetriol | — | — | — | 0.5 | 5.0 | 30.0 | — |
| Evaluation | Degree of | B | A | A | B | A | A | D |
| | melting point | | | | | | | |
| | depression | | | | | | | |
| | Yield | A | A | B | A | A | B | A |

[Mass ratio]

The results listed in Tables 1 to 3 confirmed that the addition of the alkanetriol having the number of carbon atoms of 3 to 8 to the alkanediol allowed the melting point to be lowered to an extent that the pipe clogging is effectively reduced. Setting the mass ratio of the alkanediol to the alkanetriol having the number of carbon atoms of 3 to 8 (alkanediol:alkanetriol having the number of carbon atoms of 3 to 8) in the range of 99.5:0.5 to 70:30 confirmed that an excellent melting point lowering effect was obtained and the yield of the alkanediol in the final purified product was excellent.

The sources of the components used in Examples 1 to 18 and Comparative Examples 1 to 5 are listed in Table 4.

TABLE 4

| | Number of | |
|---|---|---|
| | carbon atoms | Manufacturer name |
| 1,6-Hexanediol | 6 | KANTO CHEMICAL CO., INC. |
| 1,6,7- | 8 | Purification from |
| Octanetriol | | genetically modified E. coli |
| | | culture solution |
| Glycerin | 3 | KANTO CHEMICAL CO., INC. |
| 1,2,6- | 6 | Tokyo Chemical Industry Co., |
| Hexanetriol | | Ltd. |
| 1,2,8- | 8 | Tokyo Chemical Industry Co., |
| Octanetriol | | Ltd. |

### <Method for Producing 1,6,7-Octanetriol>

Genetically modified E. coli producing 1,6-hexanediol was prepared according to the method described in Japanese Unexamined Patent Application Publication (Translation of PCT Application) No. 2022-530467. In addition to 1,6-hexanediol, the genetically modified E. coli produces an alkanetriol as a byproduct. The genetically modified E. coli was also used in the preparation of alkanediol-containing compositions in Examples 19 to 22 described below.

Genetically modified E. coli was inoculated into a medium (carbon sources: glucose and glycerin, nitrogen source: enzyme extracts, inorganic salts: potassium phosphate and potassium hydroxide, vitamin B12, antibiotics: carbenicillin, kanamycin, and chloramphenicol, pH: 7.0, among the above components, glucose and glycerin were derived from biomass resources) sterilized with an autoclave and cultured under aerobic conditions at 30° C. for 2 to 3 hours.

Then, when the optical density of the genetically modified E. coli at 600 nm reached 0.3 to 0.6, isopropyl-β-thiogalactopyranoside and iron (II) sulfate were added so that final concentrations were 0.5 mM and 10 μM, respectively. In addition, under aerobic conditions, the genetically modified E. coli was cultured at 30° C. for 3 hours to express enzymes of the 1,6-hexanediol pathway. Subsequently, appropriate amounts of carbon sources (glucose and glycerin) were added and the inside of the incubator was placed under an atmosphere of nitrogen to form anaerobic conditions. Under these conditions, the culture was performed at 30° C. for 48 hours.

In order to obtain 1,6,7-octanetriol easily, genetically modified E. coli produced based on the method in Japanese Unexamined Patent Application Publication (Translation of PCT Application) No. 2022-530467 described in Example 6 was also used as appropriate. In that case, the medium sterilized with an autoclave not including chloramphenicol was used, glucose alone was added as a carbon source in an appropriate amount after the enzyme expression, and at the same time an appropriate amount of 6-hydroxyhexanoate was added. At this time, the inside of the incubator after enzyme expression might be either anaerobic conditions or conditions in which an appropriate amount of air was aerated.

### <<Process (a): Microfiltration>>

Cellular biomass, particulates, and high molecular weight foreign matters in the alkanediol-containing compositions were removed. In Process (a), microfiltration was performed using a spiral membrane FR (PVDF 800,000 Da) manufactured by Synder Filtration, Inc. The inlet pressure of the membrane element was maintained at 0.4 MPa. Alkanediol-Containing Composition A was obtained as a permeate.

### <<Process (b): Nanofiltration>>

Proteins, some salts, and some coloring matters in Alkanediol-Containing Composition A were removed. In Process (b), nanofiltration was performed using spiral membrane NFX (TFC 150-300 Da) manufactured by Synder Filtration, Inc. The inlet pressure of the membrane element was maintained at 3 MPa. Alkanediol-Containing Composition B was obtained as a permeate.

### <<Process (c): Ion Exchange to Remove Cations>>

Cations included in the alkanediol-containing composition B were removed. In Process (c), cation exchange was performed using a column type system. The temperature for contact with the cation exchange resin was set to 40° C. DIAION SK1BH manufactured by Mitsubishi Chemical Corporation was added to the column as a cation exchange resin, and Alkanediol-Containing Composition B was passed through the column. After passing through the column, Alkanediol-Containing Composition C was obtained.

### <<Process (d): Ion Exchange to Remove Anions>>

The anions in Alkanediol-Containing Composition C were removed. In Process (d), anion exchange was performed in a column type system. The temperature for contact with the anion exchange resin was set to 40° C. DIAION SA10AOH manufactured by Mitsubishi Chemical Corporation was added to the column as an anion exchange resin, and Alkanediol-Containing Composition C was passed through the column. After stirring, the mixture was filtered to obtain Alkanediol-Containing Composition D as a filtrate.

### <<Process (e): Process to Remove Water>>

Water in Alkanediol-Containing Composition D was removed. A thin-film distiller was used as the apparatus for Process (e). The jacket temperature was set to 70° C., Alkanediol-Containing Composition D was continuously introduced, and water was discharged by distillation from the top. Simultaneously with the discharge by distillation of water, the dehydrated Alkanediol-Containing Composition E was continuously taken out from the bottom as a bottom liquid.

### <<Process (f): Distillation Separation of Low Boiling Point Components>>

Components having lower boiling points than that of 1,6-hexanediol in Alkanediol-Containing Composition E were removed with a continuous distillation column. The Oldershaw distillation column was used as the distillation column for Process (f). Alkanediol-Containing Composition E obtained in Process (e) was continuously fed to the distillation column and the column top temperature was controlled at a constant temperature of 240° C. Continuous discharge by distillation was performed from the top of the column and continuous extraction was performed from the bottom of the column to remove the low boiling point components in Alkanediol-Containing Composition E. Alkanediol-Containing Composition F was taken out from the bottom of the column.

### <<Process (g): Distillation Separation of High Boiling Point Components>>

Components having higher boiling points than that of 1,6-hexanediol in Alkanediol-Containing Composition F were removed with a continuous distillation column. The Oldershaw distillation column was used as the distillation column for Process (g). Alkanediol-Containing Composition F obtained in Process (f) was continuously fed to the distillation column and the column bottom temperature was controlled to be constant at 260° C. High Boiling Point Component G in Alkanediol-Containing Composition F was obtained by continuous extraction from the bottom of the column. Composition H containing alkanediol was obtained from the top of the column as a column top distillate.

<<Process (h): Isolation of 1,6,7-Octanetriol from High Boiling Components Separated by Distillation>>

1,6,7-Octanetriol included in High Boiling Point Component G in Alkanediol-Containing Composition F was isolated by preparative LC. A fraction including 1,6,7-octanetriol was fractionated from High Boiling Point Component G obtained in Process (g) by a mass spectrum detection medium pressure preparative chromatography system (Smart Flash MS System) (MSD-200 manufactured by Yamazen Corporation). The eluent was evaporated from the fractionated mixture by an evaporator to give 1,6,7-octanetriol.

### Examples 19 to 22

### <Preparation of Alkanediol-Containing Compositions>

### Example 19

Genetically modified E. coli was inoculated into a medium (carbon sources: glucose and glycerin, nitrogen source: enzyme extracts, inorganic salts: potassium phosphate and potassium hydroxide, vitamin B12, antibiotics: carbenicillin, kanamycin, and chloramphenicol, pH: 7.0, among the above components, glucose and glycerin were derived from biomass resources) sterilized with an autoclave and cultured under aerobic conditions at 30° C. for 2 to 3 hours.

Then, when the optical density of the genetically modified E. coli at 600 nm reached 0.3 to 0.6, isopropyl-β-thiogalactopyranoside and iron (II) sulfate were added so that final concentrations were 0.5 mM and 10 μM, respectively. Further, the genetically modified E. coli was cultured at 30° C. for 3 hours to express enzymes of the 1,6-hexanediol pathway. Subsequently, appropriate amounts of carbon sources (glucose and glycerin) were added and the inside of the incubator was placed under an atmosphere of nitrogen to form anaerobic conditions. Under these conditions, the culture was performed at 30° C. for 8 hours.

Purification was performed by the procedures of the above <<Process (a)>> to <<Process (f)>> to give Alkanediol-Containing Composition F.

By HPLC, 1,6-hexanediol and 1,6,7-octanetriol in Alkanediol-Containing Composition F were measured and the mass ratio of these was calculated. As a result, the mass ratio of 1,6-hexanediol to 1,6,7-octanetriol was about 95:5. Few alkanediols other than 1,6-hexanediol were detected. Few alkanetriols other than 1,6,7-octanetriol were detected. This Alkanediol-Containing Composition F was used as the alkanediol-containing composition of Example 19.

### Example 20

Alkanediol-Containing Composition F was obtained in the same manner as the manner in Example 19 except that the culture time of the genetically modified E. coli under the anaerobic conditions was changed to 24 hours.

By HPLC, 1,6-hexanediol and 1,6,7-octanetriol in Alkanediol-Containing Composition F were measured and the mass ratio of these was calculated. As a result, the mass ratio of 1,6-hexanediol to 1,6,7-octanetriol was about 90:10. Few alkanediols other than 1,6-hexanediol were detected. Few alkanetriols other than 1,6,7-octanetriol were detected. This Alkanediol-Containing Composition F was used as the alkanediol-containing composition of Example 20.

### Example 21

Alkanediol-Containing Composition F was obtained in the same manner as the manner in Example 19 except that the culture time of the genetically modified E. coli under the anaerobic conditions was changed to 48 hours.

By HPLC, 1,6-hexanediol and 1,6,7-octanetriol in Alkanediol-Containing Composition F were measured and the mass ratio of these was calculated. As a result, the mass ratio of 1,6-hexanediol to 1,6,7-octanetriol was about 80:20. Few alkanediols other than 1,6-hexanediol were detected. Few alkanetriols other than 1,6,7-octanetriol were detected. This Alkanediol-Containing Composition F was used as the alkanediol-containing composition of Example 21.

### Example 22

Alkanediol-Containing Composition I was obtained in the same manner as the manner in Example 19 except that the culture time under aerobic conditions was changed to 1 hour after the genetically modified E. coli was inoculated into the medium sterilized with an autoclave and in the above <<Process (f): Distillation Separation of Low Boiling Point Components>>, Alkanediol-Containing Composition F was taken out from the column bottom of the distillation column, and thereafter the column bottom temperature of the above <<Process (g): Distillation Separation of High Boiling Point Components>> was changed to 300° C. to perform Process (g).

By HPLC, 1,6-hexanediol and glycerin in Alkanediol-Containing Composition I were measured and the mass ratio of these was calculated. As a result, the mass ratio of 1,6-hexanediol to glycerin was about 99.5:0.5. Few alkanediols other than 1,6-hexanediol were detected. Few alkanetriols other than glycerin were detected.

This Alkanediol-Containing Composition I was used as the alkanediol-containing composition of Example 22.

### <Evaluation of Degree of Melting Point Depression>

The degrees of melting point depression were evaluated in the same manner as the manner in Examples 1 to 18 and Comparative Examples 1 to 4. The results are listed in Table 5 as “Degree of melting point depression”.

### <Evaluation of Alkanediol Yield in Final Purified Product>

For each Alkanediol-Containing Composition F obtained in Examples 19 to 21, the procedures of the above <<Process (g)>> were performed to give the respective final purified products of Examples 19 to 21.

For the Alkanediol-Containing Composition I obtained in Example 22, the procedures of the above <<Process (g)>> were performed to give the final purified product of Example 22.

In Examples 19 to 21, the respective yields of the alkanediol in each final purified product were calculated from the amount of the alkanediol in each Alkanediol-Containing Composition F obtained in Examples 19 to 21 and the amount of the alkanediol in each final purified product. In Example 22, the yield of the alkanediol in the final purified product was calculated from the amount of the alkanediol in Alkanediol-Containing Composition I and the amount of the alkanediol in the final purified product obtained in Example 22. The yields calculated in each Example were evaluated according to the same evaluation criteria as the criteria in Examples 1 to 18 and Comparative Examples 1 to 4. The results are listed in Table 5 as “Yield”.

The numerical values in Table 5 represent mass ratios.

TABLE 5

Example

| Component | | 19 | 20 | 21 | 22 |
|---|---|---|---|---|---|
| Alkanediol | 1,6- | 95.0 | 90.0 | 80.0 | 99.5 |
| | Hexanediol | | | | |
| Alkanetriol | 1,6,7- | 5.0 | 10.0 | 20.0 | — |
| | Octanetriol | | | | |
| | Glycerin | — | — | — | 0.5 |
| Evaluation | Degree of | A | A | A | B |
| | melting | | | | |
| | point | | | | |
| | depression | | | | |
| | Yield | A | B | B | A |

The results listed in Table 5 confirmed that even in the case where the alkanediol-producing bacteria culture solution was used, the presence of alkanetriol having the number of carbon atoms of 3 to 8 allows the melting point lowering effect to be obtained to the extent that the pipe clogging is effectively reduced. The mass ratio of the alkanediol to the alkanetriol having the number of carbon atoms of 3 to 8 (alkanediol:alkanetriol having the number of carbon atoms of 3 to 8) within the range of 99.5:0.5 to 70:30 confirmed that an excellent melting point lowering effect was obtained and the yield of the alkanediol in the final purified product was excellent.

According to the present invention, the alkanediol-containing composition that can be used for the purification of alkanediols, can reduce the clogging of the pipes used in the alkanediol purification process, and can reduce energy required to reduce the clogging is provided.

1. An alkanediol-containing composition comprising:
an alkanediol having a melting point of 30° C. or more at normal pressure; and
an alkanetriol having number of carbon atoms of 3 to 8, wherein
a mass ratio of the alkanediol to the alkanetriol is 99.5:0.5 to 70:30.

2. The alkanediol-containing composition according to claim 1, wherein the alkanetriol comprises an alkanetriol having a boiling point higher than a boiling point of the alkanediol at normal pressure.

3. The alkanediol-containing composition according to claim 1, wherein the alkanediol comprises 1,6-hexanediol.

4. The alkanediol-containing composition according to claim 1, wherein the alkanetriol comprises at least one alkanetriol selected from the group consisting of 1,6,7-octanetriol, glycerin, 1,2,6-hexanetriol, and 1,2,8-octanetriol.

5. The alkanediol-containing composition according to claim 1, wherein the alkanediol-containing composition comprises a component produced by a living organism.

6. The alkanediol-containing composition according to claim 1, wherein the alkanediol-containing composition comprises a component derived from a biomass resource.

7. The alkanediol-containing composition according to claim 1 used for purification of alkanediols.

8. The alkanediol-containing composition according to claim 2, wherein the alkanediol comprises 1,6-hexanediol.

9. The alkanediol-containing composition according to claim 2, wherein the alkanetriol comprises at least one alkanetriol selected from the group consisting of 1,6,7-octanetriol, glycerin, 1,2,6-hexanetriol, and 1,2,8-octanetriol.

10. The alkanediol-containing composition according to claim 2, wherein the alkanediol-containing composition comprises a component produced by a living organism.

11. The alkanediol-containing composition according to claim 2, wherein the alkanediol-containing composition comprises a component derived from a biomass resource.

12. The alkanediol-containing composition according to claim 2 used for purification of alkanediols.
